(19) United States
(12) Patent Application Publication
(10) Pub. No.: US 2010/0261692 A1
(43) Pub. Date: Oct. 14, 2010

(54) METHOD OF MANUFACTURE OF COMPRESSED PHARMACEUTICAL FORMULATION CONTAINING TIBOLONE

(76) Inventors: Jaroslav Riha, Kostalov (CZ); Jaroslav Rezac, Liberec (CZ); Jan Muzikar, Praha (CZ); Rudolf Gomola, Neratovice (CZ); Vaclav Tomasek, Praha (CZ); Miroslav Trcka, Praha (CZ)

Correspondence Address: Pearl Cohen Zedek Latzer, LLP, 1500 Broadway, 12th Floor, New York, NY, 10036, US

(21) Appl. No.: 12/670,580

(22) PCT Filed: Jul. 23, 2008
(86) PCT No.: PCT/CZ08/00087

§ 371 Date: May 17, 2010

(30) Foreign Application Priority Data
Jul. 25, 2007 (CZ) PV 2007-501

(51) Int. Cl. A61K 31/57; A61P 5/30

(52) U.S. Cl. 514/178

(57) ABSTRACT

Method of manufacture of compressed pharmaceutical formulation with the active substance tibolone by direct compression into tablets, whereas during the manufacturing process the formulation is subjected to the action of a protic solvent, either by addition of 0.1 to 3% by weight of said solvent in the liquid state and/or in the vapor form by ensuring the ambient atmosphere with the contents of solvent vapors above 50% relative.

### TECHNICAL FIELD

The invention regards compressed pharmaceutical formulations containing tibolone.

### BACKGROUND ART

Products containing tibolone, chemically 17β-hydroxy-17α-ethynyl-7α-methylestr-5(10)-en-3-one, belong to the group of medicines referred to as hormone replacement therapy (HRT). These products are used in menopausal women, when the female body ceases to produce the hormone estrogen. Products alleviate symptoms related to menopause and impede the process of bone mass reduction.

During manufacture and storage of products containing tibolone a certain amount of its degradation product—isotibolone (Δ⁴-tibolone; 17β-hydroxy-17α-ethynyl-7α-methylestr-4-en-3-on) usually forms. Isotibolone is a thermodynamically stable tibolone isomer, which is formed by isomerization of the tibolone double bond. Isotibolone is also a major metabolite in the bloodstream of patients using products containing tibolone.

Thermodynamically more stable isotibolone also represents one of problematic impurities; a great effort was devoted to the formation of a stable tibolone compound, where isotobolone would form only in a limited amount.

The work of N. P. van Viet et al., Red. Tray. Chim. Pays Bas 105, 111-115 (1986) deals with the decrease of this impurity in the active substance. By the manufacturing procedure listed here tibolone with the contents of isotibolone under 1% is obtained.

In the patent application EP 1 121 375 the method of tibolone manufacture leading to a product with a very good stability is described. Through the described procedure the product with a low isotobolone contents, under 0.5%, may be obtained. Pharmaceutical products formulated from the substance manufactured using this procedure achieve isotibolone less than 1%, preferably even less than 0.7% (page 3, line 30). Pharmaceutical formulations with this impurity contents are produced from so-called basic granulate, into which tibolone is mixed and, subsequently, the mixture is compressed into tablets. This basic granulate is previously manufactured by moist granulation technique with water contents of 5.5 to 7%, preferably 6%.

In patent application WO 9847517 it was shown that product storage at higher humidity levels, i.e. 50 to 70% of relative air humidity, is advantageous for product stability. In example 5 of this application product stabilities under different storage conditions at different humidity are compared. At 25% RH the contents of isotibolone after 3 months of storage has reached 13%, whereas at 75% RH it was only 3%.

However, it has been observed that even in case of observing these conditions and employing direct compression technology, i.e. starting from a substance manufactured following EP 1 121 375, under storage conditions according to WO 9847517, there is a marked increase in tibolone impurities contents during first days after completion of pharmaceutical substance manufacturing process.

### DISCLOSURE OF INVENTION

The principle of the invention is a method of manufacture of compressed pharmaceutical formulation containing tibolone as active substance by direct tablet compression technology, where this formulation is subjected to the action of a protic solvent during manufacture. Said protic solvent action is realized either by addition of 0.1 to 3% by weight, preferably 0.5 to 1% by weight, of a protic solvent into the dry mixture during manufacturing process, or by maintaining the protic solvent vapor concentration above 50%, preferably between 50 and 65%, during preparation of the compressed formulation, or by combining both methods.

The pharmaceutical formulation thus prepared is significantly more stable immediately after manufacture completion than the formulations known so far. The pharmaceutical formulation thus prepared with the contents of 0.25 to 10% by weight of tibolone, preferably 2 to 5% by weight of tibolone, makes it possible that in standard stability tests at temperature of 25° C. and relative humidity 60±5% the contents of isotibolone does not increase by more than 0.4%, relative to tibolone peak area, within the period of 1 month. In the advantageous case it does not increase by more than 0.2%, relative to tibolone peak area, within the period of 1 month.

### DETAILED DESCRIPTION OF THE INVENTION

The method of tablet manufacture by direct compression represents a technological procedure, where the inactive substances are sieved and mixed gradually, in several steps, with the active substance, so that the result is a sufficiently homogenous mixture. This procedure does not use granulation, where, on the contrary, a solution of the bonding agent in an appropriate solvent is used, thus forming granules which are subsequently dried. The method of direct compression into tablets has the advantage of its simplicity and mildness to thermo-labile substances, which may succumb to decomposition during drying.

During experiments with direct compression of tibolone into tablets it has been shown that it is possible to prepare a composition with a very low contents of the most problematic impurity, isotibolone. When the starting active substance containing less than 0.1% of isotibolone was used, its contents immediately after processing did not increase more than to 0.2%. This value corresponds very well to the values published for alternative procedures with granulation, where the contents of isotibolone was less than 0.7%.

However, the increase in this impurity during the first days of storage of tablets manufactured by this method was a problem. For example, it has been shown that with the use of the technique of direct compression into tablets with no modifications about half the amount of the impurity formed after one entire month forms during the first 3 days of storage.

Surprisingly, it has been shown that this problem has to be solved already during pharmaceutical manufacture, and that is by exposing the material to the action of a protic solvent during the process.

Protic solvents, capable of proton (H⁺) release, may include alcohols. Mainly, lower C1 to C4 alcohols. Preferable alcohols are methanol, ethanol or isopropanol. For its universal availability and low toxicity, ethanol is the preferred one of the spectrum. An especially preferred protic solvent is water. It is a substance that is neither toxic nor flammable and is (so far) universally available.

The protic solvent may act in its liquid but also in the gaseous phase or by combination of both.

For use of the solvent in the liquid phase, 0.1 to 0.3%, more preferably 0.5 to 1.5% by weight of the protic solvent is added into the mixture during manufacture of the formulation for direct compression into tablets.

In case of use of the protic solvent in the gaseous phase the manufacture is carried out under an atmosphere containing more than 50% relative solvent vapors. The percentage relates to the other component of the atmosphere, which is usually air. The advantageous composition of the atmosphere is that with 50 to 65% relative vapors. For the purpose of use of gas phase solvent water is particularly advantageous due to facility of the procedure and its qualities mentioned above, such as non-flammability and non-toxicity.

In the following more detailed description of the ingredients used all the percentages are percentages by weight.

For the purpose of manufacture of a pharmaceutical composition according to the invention 10 to 95% of a filler chosen among lactose monohydrate, anhydrous lactose, microcrystalline cellulose, corn or potato starch, calcium hydrogen phosphate, sorbitol or mannitol is usually used.

Advantage may be taken of the disintegration properties of starch and a mixture of starch and lactose can be used. The favorable ratio is 1:1 to 1:10.

The resulting composition may contain, for example, 10 to 95% of lactose and, at the same time, 1 to 30% of starch. Instead of lactose sugar alcohols, such as mannitol or sorbitol, may be used in the given compositions. In these compositions starch acts as a disintegrant.

In case microcrystalline cellulose is used as a filler, its amount is 10 to 90%, and the disintegration properties of this substance may also be used.

Calcium hydrogen phosphate is recommended to be used in manufacture in the amount of 10 to 60%.

The composition may contain slip substances—lubricants, for example stearate-type lubricants such as magnesium stearate 0.1 to 5%.

It may contain other stabilizing agents such as antioxidants, for example ascorbyl palmitate in the amount of 0.1 to 1%.

More detailed embodiment of the procedure according to the invention is outlined in the following variations.

In the following variations of the manufacturing procedure the components, such as fillers, disintegrants, stabilizers, lubricants, are considered while bearing in mind that some substances may have multiple functions. For carrying out the process it seems to be significant in what phase and in what manner the composition is subjected to the action of the protic solvent.

### Procedure 1

Tibolone is mixed with a portion of the filler and stabilizer. In second step the formed mixture is mixed with the disintegrant and the other portion of the filler and a protic solvent in liquid phase is added to this mixture. The solvent is distributed into the entire mass as evenly as possible, which may be preferably done by sieving of the moistened mixture through a suitable sieve (for example with mesh size 0.5 to 2.0 mm) and subsequent stirring of the mixture. To the formed mixture the lubricant is added and mixed in.

### Procedure 2

Tibolone is mixed with a portion of the filler and stabilizer. The formed mixture is mixed with another portion of the filler and with the disintegrant. To this formed mixture of substances the lubricant is added and the entire mixture is homogenized. To the mixture a protic solvent in liquid phase is subsequently added. The solvent is distributed into the entire mass as evenly as possible, which may be preferably done by sieving of the moistened mixture through a suitable sieve (for example with mesh size 0.5 to 2.0 mm) and subsequent stirring of the mixture. The result is tablet substance ready to be compressed into tablets.

### Procedure 3

To a portion of the filler and disintegrant, a protic solvent in liquid phase is added. The solvent is distributed into the entire mass as evenly as possible, which may be preferably done by sieving of the moistened mixture through a suitable sieve (for example with mesh size 0.5 to 2.0 mm) and subsequent stirring of the mixture. Separately, tibolone is mixed with a portion of the filler and stabilizer. The formed mixture is mixed together with the moistened portion of the lot—filler and disintegrant. To the formed moistened mixture the lubricant is added and all the mixture is homogenized. The result is tablet substance ready to be compressed into tablets.

### Procedure 4

All the manufacturing process including compression into tablets is carried out in an environment with relative air humidity above 50%, preferably 50 to 65%. Tibolone is mixed with a portion of the filler and stabilizer. The formed mixture is mixed with another portion of the filler and disintegrant. To this formed mixture of substances the lubricant is added and the entire mixture is homogenized. The result is tablet substance ready to be compressed into tablets.

In the following section of the description all the percentages are percentages by weight if not specified otherwise.

Another aspect of the invention includes a tablet having advantageous stability characteristics, containing 0.25 to 10% of tibolone, more preferably 0.25 to 3% of tibolone, 80 to 90% of lactose monohydrate and 0.1 to 3%, preferably 0.5 to 1.5% of free water, i.e. not water bound in crystals of the solid ingredients. In a preferable embodiment this tablet contains 7 to 12% of starch for improvement of disintegration properties.

Another characteristic of the formulation according to the invention is its behavior under standard conditions of stability tests described in Pharm. Eur. The formulation of the invention containing 0.25 to 3% of tibolone, after one month of storage at temperature of 25° C. and relative humidity of 60±5%, makes it possible that the contents of isotibolone does not increase by more than 1%, more preferably 0.4%, even more preferably by 0.2%. In this case isotibolone percentage is calculated from the ratio of tibolone and isotibolone peak areas measured by standard HPLC method.

### Example 1

1. Product composition without water addition, lot 1

| | | |
|---|---|---|
| Substance name | Amount (g) | Specification |

Active substance

| | | |
|---|---|---|
| Tibolone | 250 | In-house specification |

Inactive substances

| | | |
|---|---|---|
| Lactose monohydrate | 8700 | Ph. Eur. |
| Potato starch | 950 | Ph. Eur. |
| Ascorbyl palmitate | 50 | Ph. Eur. |
| Magnesium stearate | 50 | Ph. Eur. |

Mean tablet weight 100 mg (95 to 105 mg).

2. Composition with water addition

| | | |
|---|---|---|
| Substance name | Amount (g) | Specification |

Active substance

| | | |
|---|---|---|
| Tibolone | 250 | In-house specification |

Inactive substances

| | | |
|---|---|---|
| Lactose monohydrate | 8700 | Ph. Eur. |
| Potato starch | 950 | Ph. Eur. |
| Ascorbyl palmitate | 50 | Ph. Eur. |
| Magnesium stearate | 50 | Ph. Eur. |
| Purified water | 100 | PhEur |

Mean tablet weight 100 mg (95 to 105 mg).

### Process Description:

Product Composition without Water Addition Mixture for Compression into Tablets —Lot 1

Technology of direct compression into tablets is used as the most appropriate manufacturing process. The starting materials are weighed, sieved and homogenized in three steps. During the 1st step the active substance, ascorbyl palmitate and a portion of lactose are being mixed for 15 minutes. During the second step the first portion is sieved together with the rest of lactose and potato starch. Subsequently, sieved magnesium stearate is added to the mixture and the resulting mixture is being homogenized for additional 5 minutes.

Product Mixture with Water Addition for Compression into Tablets—Lot 2

Procedure is carried out similarly as with lot 1 with the difference that before final homogenization 1% by weight of water is added to the mixture, i.e. 100 g per 10 kg of the mixture.

In both cases the mixture was compressed into round flat tablet 6 mm in diameter. Manufacturing conditions: at temperature of 20 to 25° C. and 30 to 40% RH.

### Stability Tests

### Conditions 25° C., 60±5% RH

| | Product | | | Product with | | |
|---|---|---|---|---|---|---|
| | without water | | | water addition | | |
| | addition, lot 1 | | | lot 2 | | |
| | Concentration of | | | Concentration of | | |
| | impurities - | | | impurities - | | |
| | [%]* | | | [%]* | | |
| Testing time | HT | HPT | IT | HT | HPT | IT |
| Day 0 | 0.08 | 0.02 | 0.14 | 0.03 | 0.06 | 0.11 |
| (day of lot manufacture) | | | | | | |
| Day 1 | 0.07 | 0.02 | 0.41 | 0.04 | 0.06 | 0.12 |
| Day 3 | 0.04 | 0.07 | 0.55 | 0.05 | 0.07 | 0.14 |
| Day 10 | 0.08 | 0.08 | 0.70 | 0.07 | 0.08 | 0.17 |
| Day 30 | 0.09 | 0.09 | 1.18 | 0.10 | 0.08 | 0.24 |

Abbreviations:
HT - 10-hydroxy tibolone
HPT - 10-hydroperoxy tibolone
IT - isotibolone (Δ⁴-tibolone)
*% peak areas versus tibolone peak area, detector UV DAD

### Discussion:

The data show that water substantially decreases the conversion of tibolone to isotibolone. It does not have any substantial influence on the formation of other two impurities.

### Example 2

Lot 3 had identical composition and was prepared by identical procedure as lot 1, however, during its preparation the following conditions were maintained: temperature 20 to 25° C. and relative air humidity 55 to 65% (in case of lot 1:30 to 40%).

### Stability Tests

### Conditions 25° C., 60±5% RH

| | Product lot 1 | | | Product lot 3 | | |
|---|---|---|---|---|---|---|
| | Concentration | | | Concentration | | |
| | of impurities - | | | of impurities - | | |
| | [%]* | | | [%]* | | |
| Testing time | HT | HPT | IT | HT | HPT | IT |
| Day 0 | 0.08 | 0.02 | 0.14 | 0.03 | 0.06 | 0.21 |
| (day of lot manufacture) | | | | | | |
| Day 1 | 0.07 | 0.02 | 0.41 | 0.03 | 0.07 | 0.32 |
| Day 3 | 0.04 | 0.07 | 0.55 | 0.04 | 0.09 | 0.35 |
| Day 10 | 0.08 | 0.08 | 0.70 | 0.08 | 0.10 | 0.34 |
| Day 30 | 0.09 | 0.09 | 1.18 | 0.15 | 0.09 | 0.47 |

### Discussion:

The example shows that if manufacture is carried out at higher relative humidity (55 to 65%), the conversion to isotibolone is slower under comparable conditions than at lower humidity (30 to 40%). Change in the rate of conversion into other products is not significant.

### Example 3

Lot 4 was manufactured following the same procedure as for lot 1, but during manufacture and stability testing the composition was being formed and maintained under nitrogen atmosphere with maximum water contents of 5 ppm.

### Stability Test

Lot 1 20-25° C., 30-40% RH.
Lot 4 20-25° C., nitrogen atmosphere (less than 5 ppm of water)

| | Product lot 1 | | | Product lot 4 | | |
|---|---|---|---|---|---|---|
| | Concentration | | | Concentration | | |
| | of impurities - | | | of impurities - | | |
| | [%]* | | | [%]* | | |
| Testing time | HT | HPT | IT | HT | HPT | IT |
| Day 0 | 0.08 | 0.02 | 0.14 | 0.04 | 0.02 | 0.15 |
| (day of lot | | | | | | |
| manufacture) | | | | | | |
| Day 1 | 0.07 | 0.02 | 0.41 | 0.04 | 0.03 | 0.45 |
| Day 3 | 0.04 | 0.07 | 0.55 | 0.05 | 0.04 | 0.66 |
| Day 10 | 0.08 | 0.08 | 0.70 | 0.04 | 0.08 | 2.00 |
| Day 30 | 0.09 | 0.09 | 1.18 | 0.07** | 0.09** | 2.77** |

**After 24 days.

### Discussion:

The data show that further reduction in atmospheric water during manufacture leads to an increase in the rate of conversion into isotibolone. Influence on other impurities is not significant.

### Example 4

Lot 4 was manufactured following the same procedure as for lot 2, but instead of water absolute ethanol was used in the amount of 1% by weight, related to lot size.

Composition with the addition of absolute ethanol:

| | | |
|---|---|---|
| Substance name | Amount (g) | Specification |

Active substance

| | | |
|---|---|---|
| Tibolone | 250 | In-house specification |

Inactive substances

| | | |
|---|---|---|
| Lactose monohydrate | 8700 | Ph. Eur. |
| Potato starch | 950 | Ph. Eur. |
| Ascorbyl palmitate | 50 | Ph. Eur. |
| Magnesium stearate | 50 | Ph. Eur. |
| Absolute ethanol | 100 | PhEur |

Mean tablet weight 100 mg (95 to 105 mg).

### Stability Tests

### Conditions 25° C., 60±5% RH

| | Product lot 1 | | | Product lot 5 | | |
|---|---|---|---|---|---|---|
| | Concentration | | | Concentration | | |
| | of impurities - | | | of impurities - | | |
| | [%]* | | | [%]* | | |
| Testing time | HT | HPT | IT | HT | HPT | IT |
| Day 0 | 0.08 | 0.02 | 0.14 | 0.05 | 0.02 | 0.09 |
| (day of lot manufacture) | | | | | | |
| Day 1 | 0.07 | 0.02 | 0.41 | 0.09 | 0.07 | 0.11 |
| Day 3 | 0.04 | 0.07 | 0.55 | 0.05 | 0.04 | 0.12 |
| Day 10 | 0.08 | 0.08 | 0.70 | 0.04 | 0.08 | 0.14 |
| Day 30 | 0.09 | 0.09 | 1.18 | n.a. | n.a. | n.a. |

### Discussion:

The data show that further use of ethanol during manufacture, just as the use of water, leads to a marked slow-down of the rate of conversion to isotibolone. Influence on conversion into other impurities is not significant.

1. Method of manufacture of compressed pharmaceutical formulation with the active substance tibolone by direct compression into tablets, wherein during manufacture, the formulation is subjected to an action of a protic solvent, either by addition of 0.1 to 3% by weight of said solvent in the liquid state and/or in the vapor form by ensuring the ambient atmosphere with the contents of solvent vapors above 50% relative.

2. The method according to claim 1, wherein water is used as the protic solvent.

3. The method according to claim 1, wherein an organic protic solvent chosen among C1 to C4 alcohols is used.

4. The method according to claim 3, wherein ethanol is used as the protic solvent.

5. The method according to claim 1, wherein the protic solvent is added in the liquid state to the intermediate before compression.

6. The method according to claim 5, wherein the protic solvent is added either to any of the excipients, to a mixture of excipients or to a mixture of excipients and tibolone.

7. The method according to claim 6, wherein the protic solvent is added in the liquid form directly to the directly compressible mixture and said mixture is compressed after homogenization.

8. The method according to claim 5, wherein the protic solvent is used in the amount of 0.5 to 1.5% by weight.

9. The method according to claim 2, wherein the manufacturing process of tablets is carried out at ambient relative humidity of 50 to 65%.

10. The method according to claim 1, wherein 10 to 95% by weight of the composition is formed by a filler chosen from the group consisting of lactose, lactose monohydrate, microcrystalline cellulose, potato starch, corn starch, calcium hydrogen phosphate, sorbitol, mannitol and mixtures thereof.

11. The method according to claim 10, wherein lactose or starch is used as filler.

12. The method according to claim 10, wherein the tablet contains starch and lactose in the ratio of 1:1 to 1:10.

13. Tablet obtained by method according to claim 1, wherein it contains 0.25 to 10% by weight of tibolone, 80 to 90% by weight of lactose monohydrate and 0.1 to 3% of free water.

14. The tablet according to claim 13, wherein it further contains 7 to 12% by weight of starch.

15. The tablet according to claim 13, wherein it contains 0.5 to 1.5% by weight of free water.

16. A pharmaceutical formulation in solid phase containing 0.25 to 3% by weight of tibolone, obtained by the method according to claim 1, in which after compression into tablets after one month of storage at temperature 25° C., 60±5% relative humidity, the content of isotibolone does not increase by more than 1% of the peak area of isotibolone and tibolone in standard HPLC method test.

17. The pharmaceutical formulation according to claim 16, wherein after 1 month of storage under given conditions the contents of isotibolone does not increase by more than 0.2% of the ratio of the areas.

18. The pharmaceutical formulation according to claim 17, wherein it contains 80 to 90% by weight of lactose monohydrate.

19. The pharmaceutical composition according to claim 18, wherein it further contains 7 to 12% of starch.
